(19) United States
(12) Patent Application Publication
(10) Pub. No.: US 2022/0056230 A1
(43) Pub. Date: Feb. 24, 2022

(54) POROUS CARBON STRUCTURE PRODUCTION

(71) Applicant: Neoteryx, LLC, Torrance, CA (US)

(72) Inventor: Stephen Robert Tennison, Addlestone (GB)

(21) Appl. No.: 16/990,815

(22) Filed: Aug. 11, 2020

(51) Int. Cl. C08J 9/00; A61M 1/34; A61M 1/36; B29C 43/00; C08L 25/06; C08L 61/06; C08L 97/00; C08J 9/20

(52) U.S. Cl. CPC C08J 9/0061; A61M 1/3486; A61M 1/3679; B29C 43/003; C08J 9/20; C08L 25/06; C08L 61/06; C08L 97/005; C08J 9/0028; B29C 43/006

(57) ABSTRACT

A process is provided for producing a structure into which blood or other bio-fluids can flow by capillary action, e.g. for a whole blood microsampling probe. The process comprises mixing particles of novolak resin and particles of hydrocarbon polymer, producing an uncarbonized structure from the mixture by pressurised moulding and carbonizing the moulded structure, the hydrocarbon resin being a polymer such as polystyrene that on pyrolysis has a zero carbon yield, and the particles of the hydrocarbon polymer leaving voids in the carbonized structure of sufficient size for flow of whole blood into and through the structure. The particles may be of partly cured and milled novolak resin, the novolak particles when in the moulded structure not exhibiting bulk flow during carbonization but sintering at inter-particle contact points during carbonization to provide a consolidated structure. In this variant, ethylene glycol may be used as a sintering aid. Alternatively, the particles may be of fully cured and milled novolak resin, and are mixed with the hydrocarbon polymer , the lubricant and with a binder such as lignin for providing a consolidated structure.

### FIELD OF THE INVENTION

This invention relates to a process for the production of porous carbon structures into and through which whole blood and other biological fluids may flow. Such a structure may take the form of a sampler e.g. in the form of a probe for sampling biological fluids and e.g. for the collection of samples of whole blood or other bio-fluids. Such a sampler may be used for collecting quantitative, volumetrically accurate samples of dried blood (or other bio-fluids) in a user-friendly manner e.g. as part of a microsampling device.

### BACKGROUND TO THE INVENTION

### Absorbent Probes, Pellets or Tablets

U.S. Pat. No. 10,531,821 (Rudge et al.) describes a device that is suitable as a quantitative sampling tool for biological fluids, preferably blood and is designed for samples to be easily dried, shipped, and then later analyzed. It includes an absorbent probe, preferably smaller at a distal end and larger at a fastening end, with its fastening end fastened to a holder and its other, distal end free to contact a fluid to be absorbed, such as blood. In use, the absorbent probe is placed against a blood sample or blood drop(s) and wicking action draws the blood into it. An optional barrier between the absorbent probe and holder stops blood passing to the holder or wicking to the holder. The absorbent probe is made of a material that wicks up substantially the same volume of fluid even when excess fluid is available. The volume of the absorbent probe affects the volume of fluid absorbed. A hydrophilic polymeric material was disclosed as suitable for the absorbent probe, with polyolefin indicated as especially suitable for use.

The absorbent probe was advantageously shaped with an exterior resembling a truncated cone with a narrower and rounded distal end, with the wider end fastened to the holder. Advantageously the holder had a cylindrical post that fits into a recess inside the centre of the absorbent probe and extending along the longitudinal axis of the probe and holder. Thus, the truncated conical shape had thick sidewalls that abut the post on the holder, with a distal tip joining the sidewalls and forming the distal end of the probe. The conical shape of the absorbent probe helped wick the sample quickly and uniformly. Preferred sampling time was desirably as short as possible with about 2 seconds (less if possible) being most preferred, and up to 15 seconds being acceptable for some applications. Maintaining the probe in contact with the sample blood drop for between about 2-10 seconds was thus believed sufficient, with a contact time of about 2-5 seconds preferred. The probe absorbed a predetermined volume of blood during that time, and once saturated did not absorb more blood. The size and shape of the probe could be varied to adjust the volume of absorbed blood and the rate of absorption. Blood volumes of about 7-15 μL were said to be suitable, but volumes of about 20 μL and even up to about 30 μL were indicated as desirable for some applications.

The material of probe was porous in order to absorb fluid. The internal volume of the absorbent probe material (pore volume) was preferred to be between about 30% and 50% of the total volume of the material. Additionally, the nature of the absorption required small pores (preferably cylindrical tubes although irregular shapes were also sufficient) that were nominally 20-50 m in diameter or largest cross-sectional dimension. Hydrophilic polyolefin with a density of about 0.1 to 1 g/cc was believed suitable, with preferable densities of about 0.2-0.7 g/cc, more preferably about 0.5 to 0.7 g/cc. A hydrophobic polyethylene with a non-porous density of about 0.92 g/cc which was fabricated as a porous material with a density of about 0.6 g/cc and was then plasma treated to make it hydrophilic was also believed suitable. The more easily manufactured absorbent materials were suggested to have a density of about 0.4 to 0.8 g/cc. As the density increased the time to absorb the fluid sample increased. Absorption times of about two seconds were believed suitable for blood from a live subject. The times varied with the volume of fluid sample desired and its source fluid. Materials other than polyolefin were disclosed as useful, including sintered plastics which could provide more rigidity but maintain the high absorbance rate.

Porous plastic pellets produced by Neoteryx are cone shape and are mounted on a holder via a channel in the base of the pellet. When the tip of the cone is placed in contact with the blood droplet this absorb this by capillary suction see Philip Denniff, and Neil Spooner, A Novel Dried Sample Collection Technique for Quantitative Bioanalysis, Anal. Chem., DOI: 10.1021/ac5022562⋅Publication Date (Web): 24 Jul. 2014. The amount of blood absorbed by the MITRA polymeric tips and the rate of absorption are controlled by the porosity of the tip (pore volume and pore structure) which can be produced to a close tolerance although they cannot easily be varied. The problem of hematocrit variation is overcome by allowing more time for the blood to adsorb to compensate for the higher blood viscosity. The pellets are initially white and turn red as the blood adsorbs, the process is stopped when the blood has saturated the pellet allowing for variations in the rate of absorption. After absorption of the blood and drying the pellet is sent for analysis where the dried blood is removed by washing for subsequent analysis. This approach is significantly more reproducible than the filter paper approach and the pellets can contain more blood facilitating subsequent analysis. The main design constraints on the porous polymer pellets are that the free volume in the pellet for blood absorption is typically 20 μl which can only be adjusted by changing the size of the pellet, and the pore size and pore connectivity should be such that the required volume of blood is absorbed in <5 seconds see Spooner, Volumetric Absorbtive Micro Sampling (VAMS): A Novel Dried Sample Collection Technique for Quantitative Bioanalysis (above)

US 2017/0023446 (Rietveld et al.) discloses a biological fluid sampling device, comprising an absorbent probe made of an open cell, porous carbonized material and a holder connected to the probe and configured to allow a user to manually manipulate the holder and probe during use. The probe should be of sufficient size to absorb for analysis about 1 μl to about 100 μl of blood e.g. 20 μl in about 2-5 seconds without separating the blood from plasma, the probe in an embodiment having a length of less than about 5 mm and a cross-sectional area of less than about 20 mm² with a majority of the exterior surface of the probe being exposed and available for placing against a fluid sample on a surface to absorb the sample. The thermal stability of a carbon absorbent probe is said to be very high and to provide a desirable support for several analytical conditions such as vacuum or high temperature as may arise in a mass spectrometer.

A process is disclosed for making the absorbent probes by milling phenolic resin and classifying the resulting milled particles. A sieve or mesh classifier is preferred to exclude large sized or large diameter particles and small sized particles. The resin is milled into particles that are on average 5 to 6 times the size of the pores desired in the probe. The carbon-based probe is said to be usable for biological purposes (especially blood) with average pore sizes as low as 10 μm and as large as 150 μm, with other pore sizes suitable for other applications. Average pore size may be determined by bubble point testing using air. The pmresin particles for a 40 μm pore size preferably have a one sigma distribution with 65% of the resin sized between about 160 μm and 240 μm for a 200 μm resin particle. Similarly, for a 150 μm pore size the resin preferably has about 65% of the resin sized between 750-1080 μm, using sieve classification to size the particles. A preferred way to make the absorbent probe is believed to be pressing the resin into shape by powder compaction to form a probe-blank and then pyrolize that probe-blank, after which the pyrolized probe may undergo undergoes a process to increase its wettability or hydrophilic properties e.g. by treatment with an oxygen plasma or treatment with plasma enhanced CVD . A suitable resin is said to be as described in U.S. Pat. No. 8,227,518 (Tennison et al.).

### Porous Carbon Structures

Methods have previously been developed for making carbonised and optionally activated shaped structures (monoliths) from phenolic resin precursors. Monolithic or shaped structural porous carbon can be made by partially curing a phenolic resin to a solid, comminuting the partially cured resin, extruding the comminuted resin, sintering the extruded resin so as to produce a form-stable sintered product and carbonising and activating the form-stable sintered product. EP 0 254 551 (Satchell et al., also U.S. Pat. No. 4,917,835, the contents of which are incorporated herein by reference) gives details of methods of forming porous resin structures suitable for conversion to porous carbon structures. WO 02/072240 (Place et al., now U.S. Pat. No. 6,964,695, the disclosure of which is incorporated herein by reference) gives further details of producing monolithic structures using sintered resin structures of EP 0 254 551.

In a variant of this process for producing carbon monoliths, the resin cure is controlled so that it is sufficient to prevent the resin melting during subsequent carbonisation but low enough that the resin particles produced during the milling step can sinter during subsequent processing. The amount of crosslinking agent and the temperature and duration of the partial curing step are selected as to give a degree of cure sufficient to give a sinterable product, and such that a sample of the partially cured solid when ground to produce particles in the size range 106-250 μm and tableted in a tableting machine gives a pellet with a crush strength which is not less than 1 N/mm. Preferably the pellet after carbonisation has a crush strength of not less than 8 N/mm. The comminuted resin particles may have a particle size of 1-250 μm, in embodiments 10-70 μm.

Carbonisation takes place preferably by heating above 600° C. for the requisite time e.g. 1 to 48 hours and takes place under an inert atmosphere or vacuum to prevent oxidation of the carbon. On carbonisation the material loses about 50% weight and shrinks by about 50% volume but, provided the resin cure stage was correctly carried out, this shrinkage is accommodated with little or no distortion of the monolith leading to a physical structure identical to that of the resin precursor but with dimensions reduced by ˜30%. The inter-particle void size is also reduced by ˜30% although the void volume (ml/ml) remains essentially unaltered. During carbonisation the microstructure of the porous carbon develops, particularly at temperatures above 600° C. After carbonisation there may be partial blocking of the microstructure by the decomposition products from the carbonisation process. These blockages may be removed by activation to provide rapid access to the internal structure of the carbon that may be desirable for the operation of the monoliths as adsorption devices.

This production route is limited to the use of novolak resins and this in turn limits the pore structure that can be produced to the approximately 1 nm pores that are characteristic of all novolak derived carbons and larger macropores, typically greater than 1 μm, that are produced by the voids between the sintered particles. It is not possible by this route to produce products with pores in the large meso-small micro range of sizes.

In US 2013/0072845 (Tennison et al., now U.S. Pat. No. 9,278,170) a method is described for extending the porosity of the above structures to include meso and or small macro pores in addition to the micropores that derive from the novolak resin. In this invention solid particles of a first phenolic resin which is partially cured so that the particles are sinterable but do not melt on carbonisation are mixed with particles of a second phenolic resin that has a greater degree of cure than said first phenolic resin and has a mesoporous and/or macroporous microstructure generated by solvent pore forming that is preserved on carbonisation; forming the mixture into a dough; extruding the dough to form a shaped product and stabilizing its shape by sintering.

In this production route the secondary, highly cured, meso/macro porous resin component is not strongly bound into the structure due to its high degree of cure but rather is trapped in a cage formed by the sinterable resin component. This approach limits the amount of the second material than can be incorporated due to the requirement to form the cage structure. This leads to a reduction in strength when compared to the materials produced entirely from the sinterable resin particles. The extent of the larger pore structure is also limited by dilution of the matrix with the first resin component. This production route can also be used with second components other than phenolic resin such as activated carbons but in this instance differential shrinkage between the particles comprising the cage and the second component during the pyrolysis process leads to stress cracking and a further reduction in mechanical strength. Thus whilst it is possible to produce complex shapes the reduced meso/macro pore capacity and strength limits there used in demanding applications such as blood filtration.

The production of the large meso/small micro pore carbons is described in U.S. Pat. No. 8,383,703 (Tennison et al., 2103) which is incorporated herein by reference. The preferred route for producing these materials is through the use of pore formers where ethylene glycol is the preferred component although other solvents may also be used. These meso/macro porous resins can be produced either as beads or as powders. In the routes described in US 2008/025907 (Tennison et al., now U.S. Pat. No. 7,842,736) the precursors, typically comprising the novolak resin and the curing agent (typically hexamethylenetetramine (HTMA)) are dissolved in the pore forming solvent (typically ethylene glycol) in the ratios necessary to generate the required pore structure and degree of cure. The mixture can either be cured by dispersing in hot oil to form beads or placed in trays and cured in an oven. In the latter case the block of cured resin is subsequently processed by milling to give either the finished powder or a precursor for extrusion. The limitation of this route is the strength, attrition resistance and control of porosity in monolith materials produced by this route is insufficient to allow these materials to be used in demanding applications such as blood filtration.

WO 2017/009662 (Tennison), see also US 2019/0022623 (U.S. Pat. No. 15/745399) incorporated herein by reference disclose providing a carbonised and optionally activated monolith structure comprising meso/macro porous carbon particles derived from a fully cured phenolic novolak resin exhibiting mesoporosity bound with 10-40% by weight of carbonised lignin, the carbonised monolith exhibiting micropores of diameter <2 nm and mesopores of diameter 2-50 nm with voids of size >1 μm between the particles defining paths for fluid to flow into and through the structure. The carbon particles may be derived from milled meso or microporous resin, and the structure may have a mixture of micropores of approximately 1 nm and voids of between 2 and 500 nm.

The above structure may be produced from a first component which is particles of a fully cured phenolic novolak resin exhibiting mesoporosity with mesopores of diameter 2-50 nm and 10-40% by weight of a second component which is high purity water insoluble powdered lignin, by a process comprising mixing the first and second components, producing an uncarbonised monolith structure from the mixture by extrusion or pressurised moulding, and forming from the uncarbonized monolith structure a carbonised and optionally activated monolith structure as aforesaid. The monolith may be carbonised at a temperature of at least 700° C. in an inert gas and activated by treatment in flowing carbon dioxide at a temperature of at least 850° C., preferably 900° C. for a time selected to give a required weight loss of which may be at least 20%, preferably 25%.

In a further embodiment, the pore structure of the novolak resin can be further modified by incorporating the lignin into the novolak by dissolving both components into the pore former e.g. ethylene glycol. Thus, the cured novolak resin may be produced by (a) dissolving a water insoluble lignin and novolak resin in ethylene glycol; (b) dissolving hexamethylenetetramine (HMTA) curing agent in ethylene glycol; (c) mixing the two solutions to give at least 15 and preferably 20 parts of HMT per 100 parts of resin solids (lignin+novolac), the ratio of the total solids (lignin+novolak+HMT) to ethylene glycol and of lignin to novolac being selected according to the required macroporosity. The mixed solution may be poured into a tray, cured at 150° C. for 2 hours and then comminuted to form powder. Thus, a block of cured resin may be granulated to give particles of approximately 1 mm, after which the ethylene glycol may be removed from the granulated cured resin by either washing in hot water or vacuum drying. The ethylene glycol-free granulated resin may be jet milled to provide a powder with at least 90% of the powder <100 μm, e.g. the particles may have a mean size of approximately 40 μm.

The lignin may be a product of the Organosolv process. Particles of lignin may have a D90 of less than 100 μm and a mean particle size of approximately 40 μm. The content of the lignin component may be about 25% weight. Solid particles of a pure water insoluble lignin powder may be mixed with particles of the cured meso/macro porous phenolic resin, after which the mixture may be formed into a dough and pressure moulded to form the shaped product. Thus, forming the dough may be by mixing the first lignin component with the milled resin particles and with methyl cellulose, PEO and water, and optionally other additives to control the rheology the dough.

Alternatively the cured mesoporous resin particles with a size <100 um can be dry mixed with about 25% wt of the lignin binder with a particle size of <100 um along with <2% wt of a lubricant, e.g. stearic acid, and then pelleted using a single station or a multi station rotary pelleting machine.

### SUMMARY OF THE INVENTION

A problem with biological fluid sampling devices based on carbon is the provision of moulded and carbonized structures suitable for use as absorbent tips of such devices which have internal spaces or voids into which acceptable quantities of fluids such as whole blood which includes red blood cells of size 6-8 μm and thickness at the thickest point of 2-2.5μm rather than merely a liquid component thereof such as plasma can flow by capillary action. It has now been realized that certain hydrocarbon polymers when mixed in the form of particles with particles of novolak resin to be carbonized can be sufficiently thermally stable e.g. melting and decomposition resistant to remain in place until the onset of carbonization of the novolak, but decompose with zero carbon yield leaving voids in the resulting carbonized structures of sufficient size and connectedness to permit enhanced capillary flow, in embodiments offering an increased uptake of fluid to be sampled and an increased rate of uptake. As explained in US 2017/0023446 (Rietveld et al.) the increased thermal stability of the carbonized structures permits analysis by an increased range of techniques e.g. thermal desorption mass spectrometry

In one aspect, the invention provides a process for producing a structure into which blood or other bio-fluids can flow by capillary action, said process comprising:

mixing particles of novolak resin and particles of hydrocarbon polymer;

producing an uncarbonized structure from the mixture by pressurised moulding; and

carbonizing the moulded structure at or above 400° C., e.g. at 700 to 900° C., in either carbon dioxide or nitrogen, where the hydrocarbon polymer on pyrolysis has a zero carbon yield, and the particles of the hydrocarbon polymer leaving voids in the carbonized structure of sufficient size for flow of whole blood into and through the structure.

For the production of simple bi-modal carbonized tablets, probes or like structures according to the above process, the particles may be of partly cured and milled novolak resin, the novolak particles when in the moulded structure not exhibiting bulk flow during carbonization but sintering at inter-particle contact points during carbonization to provide a consolidated structure. Prior to moulding and carbonization, the ability of the particles to sinter may be increased by treatment with a hydroxy modifier e.g. ethylene glycol, propylene glycol or another polyol sintering aid, ethylene glycol being preferred. For multi-modal carbonized tablets, probes or like structures produced according to the above process wherein the particles may be of fully cured and milled novolak resin, and are mixed with the hydrocarbon polymer, the lubricant and with a binder for providing a consolidated structure. The milled particles may be the result of (a) dissolving a water insoluble lignin and novolak resin in ethylene glycol; (b) dissolving hexamethylenetetramine (HMTA) curing agent in ethylene glycol; (c) mixing the two solutions to give at least 15 parts of HMTA per 100 parts of lignin+novolak, the ratio of the lignin+novolak+HMTA to ethylene glycol and of lignin to novolak being selected according to required macroporosity. For curing, the mixed solution may be poured into a tray, cured at 150° C. for 2 hours and then comminuted to form particles. Thus, the block of cured resin e.g. from the tray may be granulated to give particles of approximately 1 mm, after which the ethylene glycol is removed from the granulated cured resin by either washing in hot water or vacuum drying. The ethylene glycol-free granulated resin is then jet milled to provide a powder with at least 90% of the powder <100 μm.

In the above process, moulding and carbonization may be employed to produce a carbonized of moulded structure of an open-celled porous carbonized material shaped as an absorbent probe and in embodiments suitable for use with devices that can manipulate a pipette tip. Advantageously the probe which in embodiments is of frustoconical shape and with a pore volume of at least 35% is of sufficient size and porosity to absorb for analysis about 1 μl to about 100 μl of whole blood e.g. 5-20 μl in about 2-5 seconds without separating the blood from plasma. In embodiments, the probe has a length of less than 5 mm, a cross-sectional area of less than 20 mm² and a flat surface that in use is exposed and available for placing against a fluid sample on a surface to absorb the sample.

### DETAILED DESCRIPTION OF PREFERRED EMBODIMENTS

### Types of Pore in Carbonized Structures

As used herein, the term “microporous” refers to a carbon or other material possessing pores with diameter <2 nm, as measured by nitrogen adsorption methods and as defined by IUPAC.

As used herein, the term “mesoporous” refers to a carbon or other material possessing alongside micropores, pores with diameter from ca. 2 nm to ca. 50 nm, as measured by nitrogen adsorption and mercury porosimetry methods and as defined by IUPAC.

As used herein, the term “macroporous” refers to a carbon or other material possessing alongside micropores and mesopores , pores with diameters larger than 50 nm. This is preferably measured by mercury porosimetry methods and as defined by IUPAC, comprises pores of greater than 50 nm diameter.
By “monolithic” is meant that the resulting porous carbon is in a single piece i.e. not granular or not composed of granular carbons bound together by a binder, for instance a polymer etc. and is composed entirely of carbon. In relation to monoliths, inter-particle voids are spaces formed within a structure that has been created from solid fully cured resin particles and lignin and carbonising and activating the resulting structure. Such voids may have sizes of the order of about 5 μm but typically is ˜20% of the size of the powdered resin used to produce the monolith.

### General Comments

The variables and their impact are summarised below:
1. Resin production—the cure conditions for the novolak resin fix the sintering capability of the cured resin powder. The standard route uses a cure temperature/time of 150° C. for 1 hour. Lower degrees of cure could be beneficial in terms of formed pellet strength crush strength by enhancing sintering. Homogeneity of cure is also important.
2. Milled resin particle size and distribution. The pore volume in the pellet is reasonably constant across a wide range of resin powder particle sizes except at the limits of very large and small mean powder sizes. At these limits this tends to reflect the way in which the powder fills the pelleting die rather than any intrinsic resin property. The production testing has tended to focus on a mean particle size of ˜70 microns. Such milled resins gives rise to pellets with a mean size pore measured using mercury porosymetry that is approximately 20% of the primary resin particle size. For resin powders with a mean particle size of 70 μm this equals ˜14 μm, and a pore volume of typically 30-40% defined by the voids between the carbon particles. Increasing the particle size reduces the compressive strength of the tablets.
3. Powder production—For larger scale pellet production powder production involves milling and possibly classification (removal of fines). This has been achieved with a classifying jet mill but other mills could be used provided control of the mean particle size is achieved. The effect of fines from the milling process is complex. The presence of fines in the resin powder can significantly improve the strength of the formed pellets but also reduces the pore volume and the rate of blood absorption.
4. Tablet production—This has been done with two presses—a small single station hydraulic press where the powder was loaded by weight which gave excellent reproducibility and a multi-station rotary press. The single station press gave independent control of the tablet pressure (P), time (T) and the size of the tablet though the amount loaded. This demonstrated that P and T had no significant effect on the tablet density which indicates that they had no effect on the void volume and demonstrated that it should be possible to use a multi-station press where there is no way of adjusting the pressure and residence time. However the pressure did impact on the blood absorption properties which suggests that it had an impact on the pore structure in a subtle way beyond the simple pore volume. In the rotary press the tablet volume was fixed by gravity filling which gave significant problems with reproducibility.
5. Pyrolysis—the resin tablets were carbonised in either flowing nitrogen or carbon dioxide at temperatures from 500 to 900° C. Temperature had a significant effect on the blood properties. Whilst most of the work was carried out at 700° C. there is evidence that lower temperatures could have a beneficial effect on the blood absorption properties but these have detrimental effects on the tablet mechanical properties. There is also evidence that use of CO₂ is preferable to nitrogen
6. Polystyrene addition—the blood uptake is a function of the pore volume within the formed tablet. For the simple resin tablets this limits the uptake to 20-25 μg, approximately 35% volume, (significantly less than this for extruded tablets where the dough production process leads to closer packing of the resin powder and lower pore volumes). The pore volume can however be increased through the addition of a polymeric pore former. This is added to the cured resin powder during the forming process During tablet pyrolysis this then decomposes completely to give a zero carbon yield leaving void spaces in the pellets. Polystyrene (PS) is the preferred pore former but other polymers with a zero carbon yield on pyrolysis could be used. There are two main variables—the amount of added pore former and the particle size of the pore former. The addition of the PS does significantly increase the pore volume and therefore the blood uptake with weight uptakes up to 50 μg combined with fast blood absorption BUT at the cost of deterioration in mechanical properties. This is not unexpected as the mechanical strength of any ceramic material is inversely dependent on the porosity.

### Novolak Resins

In addition to the control of structure that can be achieved with phenolic resin it has a further benefit in that the pyrolysis yield is higher than nearly all other precursors, natural or synthetic, and for that reason has been very widely used in composite materials. When any organic precursor, natural or synthetic is heat treated in an inert atmosphere it undergoes a significant transformation that varies with the precursor. Cellulosic materials, which have very high hero atom contents with only 51wt % carbon, lose water via the OH groups on the rings and methanol from the side chains along with larger and more complex fragments leading to the formation of a coalesced ring structure. The typical weight yield from carbonisation is a maximum of ˜20% weight.

Phenolic resin is very different in that it has a very low hetero atom content and pyrolysis of their structure typically gives rise to a 50% weight yield with the pyrolysis loss being largely due to the loss of entire phenolic fragments from short chains within the structure. The tangled nature of the resin structure also means that coalesced ring structures that gives rise to the laminar pore structure in conventional polymer derived carbons do not form. The resultant structure after carbonization shows no evidence for any porosity or 3-dimensional ordering and a structure that is referred to as “glassy” or amorphous. What is more significant are the larger nanostructures that become apparent after carbonisation e.g. a condensed globular structure with domains of <40 nm, typically 10-20 nm.

Resins for making carbonaceous material can be prepared from any of the starting materials disclosed in WO 02/12380 (Kozynchenko et al., Nucleophilic components may comprise phenol, bisphenol A, alkyl phenols e.g. cresol, diphenols e.g. resorcinol and hydroquinone and aminophenols e.g. m-amino-phenol.

It is preferred to use as nucleophilic component a phenolic novolak or other similar oligomeric starting material which, because it is already partly polymerized, makes the polymerization to the desired resin a less exothermic and hence more controllable reaction.

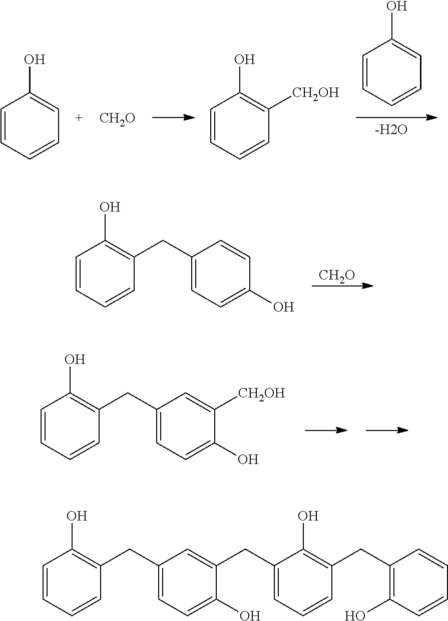

The preferred novolaks have average molecular weights (AMW) in the range of from 300 to 3000 prior to cross-linking, corresponding to a degree of polymerisation (DP) with respect to phenol of about 3-30 and may be solids with melting points in the region of 100° C. They are thermally stable and may be melted and reset without any further reaction occurring. They require the addition of a curing agent to cross link these chains to produce the final resin. In contrast, resols are base catalysed with a phenol:formaldehyde ratio of >1. The reaction produces a “treacly” resin which is sold as either an aqueous or methanolic solution. Resols are terminated by mutli-hydroxyl phenol groups which are still reactive and the product needs to be kept at <0° C. or it will continue to react to produce the cross linked resin. Curing is simply by the application of heat and results in a fully crosslinked thermoset resin. It is therefore impossible to control the degree of crosslinking.

Novolak resins of average molecular weight less than 2000 and preferably less than 1500 are thermally stable in that they can be heated so that they become molten and cooled so that they solidify repeatedly without structural change. They are cured on addition of nucleophilic cross-linking agents and heating the most common of which is hexamethylene tetramine (HMTA) The Fully cured resins are infusible and insoluble.

The nucleophilic component may be provided alone or in association with a polymerization catalyst which may be a weak organic acid miscible with the novolac and/or soluble in the pore former e.g. salicylic acid, oxalic acid, phthalic acid orp-toluene sulfonic acid.

The novolak resins can be further modified to introduce mesopores into the resin structure through the use of a pore former. In this instance the novolak resin and the cross linking agent are dissolved in ethylene glycol. The concentration of novolak in the pore former may be such that when combined with the solution of cross-linking agent in the same pore former the overall weight ratio of pore former to (novolak+crosslinking agent) is at least 125:100 by weight. The actual ratios of novolak:pore former and crosslinking agent:pore former are set according to convenience in operation e.g. in the case of the process disclosed in WO 2008/043983 (Tennison) by the operational requirements of a bead production plant and are controlled by the viscosity of the novolak:pore former solution such that it remains pumpable and by the ratio of crosslinking agent:pore former such that the crosslinking agent remains in solution throughout the plant.

### Pore-Formers for Mesoporous Resin Particles

For mesoporous resin production, the pore former also acts as solvent. Thus, the pore former is preferably used in sufficient quantities to dissolve the components of the resin system, the weight ratio of pore former to the total components of the resin system resin being preferably at least 1.25:1.

Details of suitable pore formers are given in WO 02/12380 (Tennison). The pore former may be, for example, a diol, a diol-ether, a cyclic ester, a substituted cyclic or linear amide or an amino alcohol e.g. ethylene glycol, 1,4-butylene glycol, diethylene glycol, triethylene glycol, γ-butyrolactone, propylene carbonate, dimethylformamide, N-methyl-2-pyrrolidinone and mono ethanolamine, ethylene glycol being preferred, and where the selection is also limited by the thermal properties of the solvent as it should not boil or have an excessive vapour pressure at the temperature used in the curing process.

It is thought that the mechanism of mesopore and macropore generation within the individual particles of resin is due to a phase separation process that occurs during the cross-linking reaction. In the absence of a pore former, as the linear chains of pre-polymer undergo cross-linking, their molecular weight initially increases. Residual low molecular weight components become insoluble in the higher molecular weight regions causing a phase separation into cross-linked high molecular weight domains within the lower molecular weight continuous phase. Further condensation of light components to the outside of the growing domains occurs until the cross-linked phase becomes essentially continuous with residual lighter pre-polymer trapped between the domains. In the presence of a low level of pore former the pore former is compatible with, and remains within, the cross-linked resin domains, (e.g., <120 parts/100 parts Novolak for the Novolak-HMTA-Ethylene Glycol reaction system), whilst the remainder forms a solution with the partially cross-linked polymer between the domains. In the presence of higher levels of pore former, which exceed the capacity of the cross-linked resin, the pore former adds to the light polymer fraction increasing the volume of material in the voids between the domains that gives rise to the mesoporosity and optionally macroporosity. In general, the higher the pore former content, the larger the wider the mesopores up to macropores and the higher the pore volume.

This phase separation mechanism provides a variety of ways of controlling the pore development in the cross-linked resin structures. These include chemical composition and concentration of the pore former; chemical composition and quantity of the cross-linking electrophilic agents, presence, chemical nature and concentration of modifying nucleophilic agents, chemical composition of phenolic nucleophilic components (phenol, novolac), presence, chemical nature (acidic, basic), the presence of water within the solvent and concentration of any curing catalyst if present.

Both protic and aprotic solvents of different classes of organic compounds match these requirements and can be used as pore formers, both individually and in mixtures. In addition to dissolving the reactive components and any catalyst, the pore former should also, in the case of phenolic resins, be compatible with water and/or other minor condensation products (e.g. ammonia) which are formed by elimination as polymerization proceeds, and the pore former is preferably highly miscible with water so that it can be readily removed from the polymerized resin by washing.

### Production of Mesoporous Resin Precursor

For the purposes of monoliths or other structures WO 02/12380, discloses the production of the resin in powder rather than bead form. For the production of shaped structures, such as monoliths, a resin powder is required with a mean particle size of 20 to 100 μm, preferably around 40-70 μm. This may be manufactured by producing a mixed solution of the resin component and the cross-linking agent in the pore forming solvent. This typically comprises a medium molecular weight novolak, although the molecular weight is not critical, and HMTA dissolved in the pore forming solvent, preferably ethylene glycol (EG). The two solutions, HMTA:EG and Novolak:EG are produced separately as they can be heated to enhance the dissolution. A critical requirement for the pore forming solvent is that it dissolves both the resin and crosslinking agent. The two cold solutions are then mixed. The novolak:HMTA composition is 100 parts:20 parts whilst the ratio of EG: HMTA+novolak is increased to increase the meso/macro pore volume. The mixed solution is then cured which requires a temperature of approximately 150° C. This can either be in batch mode, where the mixture is poured into trays and then placed into a preheated oven, and which gives rise to blocks of cured resin which then require granulation, or in a continuous rotary oven where the product may be large granules. The glycol can be removed by washing or vacuum drying and the particle size of the cured resin for washing is preferably a 1-2 mm which can be produced by granulation.

### Lignin

One problem which arises is how to bind highly cured porous novolak resins to give a high strength, attrition-resistant structure.

It was surprisingly found that lignin, which is essentially a naturally occurring phenolic resin, can be used as a binder phase with a wide range of second phases. In marked contrast to the problems experienced when using pitch as a binder, as is normally used in commercial activated carbon processes, the use of lignin produces form-stable green materials which show little or no distortion on firing. In marked contrast to the tars or pitches normally used as binders in active carbon production, lignins can be used as binders for a wide range of nanoporous precursors, significantly expanding the range of controlled structure materials that can be produced. Without being bound by this explanation we believe that the viscosity, melt flow and carbonisation characteristics of the lignins allows them to bond cured novolak particles together without excessive flow which would infiltrate the pore structure of the second phase and would also disperse throughout the matrix giving poor bonding. These materials then convert to carbon with a high yield of typically around 30% without the addition of any curing agents. These lignin binders can be beneficially used with second phases including controlled meso/macro structure phenolic resins.

The lignins have the further advantage that, as essentially naturally occurring phenolic resins based on coniferyl alcohol, sinapyl alcohol and p-coumaryl alcohol shown below.

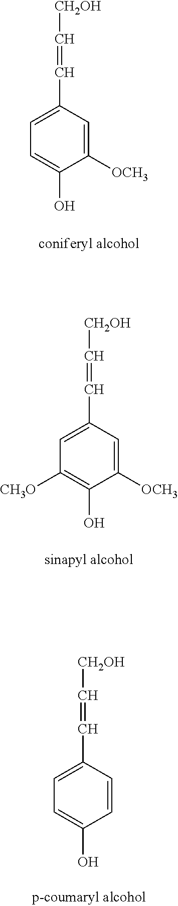

they are compatible and co-soluble with the novolak resins used in the formation of other phenolic resin derived carbons. They cannot be crosslinked in the same way as novolak resins which means that in their pure form they cannot be easily converted to controlled meso-macro pore structure carbons as they melt and foam during pyrolysis. However, when processed as a mixture with a standard Novolak resin the melting characteristics of the lignin are suppressed allowing the production of complex shaped and pore structure products, even with low levels of the second phase material.

If the higher plants are considered there are two main types of lignin where these precursor molecules are present in different proportions depending on the organic precursor. Hardwoods comprise a mixture of coniferyl and sinapyl building blocks whilst softwood lignin comprises more than 90% coniferyl alcohol. The resulting lignin is essentially a naturally occurring, medium molecular weight phenolic resin where the thermoplastic behaviour is a function of the composition which in turn depends on the precursor. As such it gives a high carbon yield on pyrolysis which is critical to achieve the good binder performance we have observed. Surprisingly however the binder performance is significantly enhanced when compared with synthetic phenolic resins of similar molecular weight. Whilst not wishing to be bound by the explanation the applicants believe that this is due to different melt flow characteristics of the two materials. As such the performance of the lignin materials in the processes described in this specification will be influenced by the melt flow and therefore by the precursor material.

At present lignin is generally a waste by-product of bio-refining and paper making where cellulose is generally the desired component. In paper making by the Kraft process lignin is generally produced as the acidic black liquor although it is also produced as a relatively pure material by for instance the Organosolv process (U.S. Pat. No. 3,585,104 Kleinert) which is used to extract the lignin in solution which is then precipitated. The lignin from the traditional Kraft process cannot be used in the process of this invention due to the high level of contaminants but more critically its water solubility which precludes the use of a conventional aqueous extrusion processes. The preferred material is produced by processes such as Organosolv and is a high purity water-insoluble powder that can be used in extrusion and other forming processes. In particular its metal content is low, preferably only a few ppm.

According to the Kleinert patent the Organosolv process involves digesting subdivided fibrous plant material in a digester at an elevated digesting pressure and at an elevated digesting temperature without pre-impregnation of pulping agent. Kleinert explains that aqueous mixtures of the lower aliphatic alcohols, such as methanol, ethanol, propanol, and aqueous mixtures of the lower aliphatic ketones, such as acetone, or aqueous mixtures containing both lower aliphatic alcohols and lower aliphatic ketones are appropriate pulping agents, aqueous mixtures of ethanol in the range 20%-75 wt % ethanol being preferred. The claimed process comprises:

(a) feeding said subdivided fibrous plant material to an inlet of said digester and moving said fibrous plant material through the digester to a fibrous plant material outlet remote from said inlet;

(b) introducing a liquid pulping agent into said digester at a point intermediate the fibrous plant material inlet and the fibrous plant material outlet, said pulping agent being at a temperature corresponding essentially to said digesting temperature, and being an aqueous mixture of a member selected from the group consisting of a lower aliphatic alcohol, a lower aliphatic ketone and their mixtures and containing about 20-75 wt % of said member;

(c) flowing said pulping agent in counter current contact with said fibrous plant material, heating said fibrous plant material to substantially said elevated digesting temperature substantially immediately on its being fed into said digester, and dissolving non-cellulosic water-soluble components of said fibrous plant material in said pulping agent on its being contacted with said pulping agent;

(d) withdrawing pulping agent containing said non-cellulosic components from said digester at a point adjacent said subdivided fibrous plant material inlet, said withdrawn pulping agent having a temperature corresponding substantially to said elevated digesting temperature so that no appreciable cooling of the withdrawn pulping agent occurs; and

(e) withdrawing digested fibrous plant material from said digester through said fibrous plant material outlet.

An advantage for this invention is that the cost of the lignin, essentially a waste material, is lower than synthetic oil derived phenolic resins. It can also be available in very large quantities. A further advantage of lignin in this invention is that as it is essentially a naturally occurring phenolic resin the carbon yield during pyrolysis is high, typically around 30%. This is lower than that achieved with the synthetic phenolic resin, primarily reflecting loss of the side chains, but is none the less significantly higher than the <20% yield typically achieved with cellulosic precursors. The applicants believe that all water insoluble lignins will be usable in the processes of the current invention but that some benefits may be achievable through the use of different lignins with selected melt flow characteristics depending on the proposed end use, whether as binder, structure modifier or the main structure forming component.

30

### Cross-Linking Agents for Novolak Resins

The cross-linking agent is normally used in an amount of from 5 to 40 parts by weight (pbw) per 100 parts by weight of the nucleophilic components e.g. novolak, typically from 10 to 30 (e.g. 10, 15 or 20) pbw cross-linking agent per 100 pbw of nucleophilic component. It may be, for example, an aldehyde e.g. fonnaldehyde or furfural or a polyamine e.g. HMTA (hexamethylene tetramine), melamine or hydroxymethylated melamine. HMTA is preferably used as cross-linking agent.

For a partially cured and sinterable resin material there may be employed up to 5 pbw of HMTA per 100 pbw of novolak. However, for the production of a mesoporous/macroporous resin it is essential that the resin is fully cured. HMTA or other cross-linking agents are preferably used at a proportion of 15 to 25 pbw. Whilst the stoichiometric amount required for complete curing is approximately 15%, a level of 20% is preferably used to guarantee full curing. This ensures formation of the solid resin with maximal cross-linking degree and ensures the stability of the mesopore structure during subsequent removal of the pore former. At lower degrees of cross linking the structure tends to collapse during removal of the pore former prior to pyrolysis.

A current process for producing the sinterable cured resin involves placing the milled novolak/5% weight HMTA powder in trays (˜50 cm square) to a bed depth of ˜3cm. The trays are lined with “TygerFilm—glass reinforced Teflon) to prevent the molten resin sticking to the tray. The current process uses a carefully controlled temperature profile and comprises 5 steps:

(a) heat from ambient to 100° C. over 1 hour. The resin does not melt during this stage.

(b) The resin is held at 100° C. for 1 hour. The purpose is to remove adsorbed moisture as varying levels can impact on the final cure

(c) During the next stage—heating to the final cure temperature (typically 150° C.). The resin melts at around 120° C. and the hex dissolves in the molten resin. It is in this dissolved stage that the hex decomposes and the cure reaction commences.

(d) The resin, by now in a solid state, is held at the final cure temperature for 2 hours after which the oven is allowed to cool back to room temperature.

For the production of the meso/macro porous resin two solutions, one of the novolak resin dissolved in ethylene glycol and the other of HMTA dissolved in ethylene glycol are prepared such that when mixed the combined solution has the required Novolak:HMTA:ethylene glycol to provide the required pore structure. The mixed solution is then poured into trays (˜50 cm square) to a bed depth of ˜3 cm. The trays are lined with “TygerFilm”—glass reinforced Teflon) to prevent the molten resin sticking to the tray. These trays are then placed directly into a preheated oven at 150 C and held for 2 hours.

Once the trays have cooled the resin blocks are granulated to give approximately lmm particles after which the ethylene glycol pore former is removed either by washing with hot water or by vacuum drying. The resulting mesoporous resin, free from ethylene glycol, can then be milled to the required particle size for subsequent pelleting, typically 70 microns, and optionally classified to remove fines of <20 μm.

### Materials for Macropore Formation—Polystyrene

Blood uptake by a carbonized moulded structure is a function of the interparticle pore volume within the formed tablet. For simple resin tablets this limits the uptake to 20-25 μg, equivalent to a pore volume of approximately 35% (significantly less than this for extruded tablets where the dough production process leads to closer packing of the resin powder and lower pore volumes). The pore volume can however be increased through the addition of a polymeric pore former. This is added to the cured resin powder during the forming process During tablet pyrolysis this then decomposes completely to give a zero-carbon yield leaving void spaces in the pellets. Polystyrene is the preferred pore former but other polymers with a zero-carbon yield on pyrolysis could be used. There are two main variables—the amount of added pore former and the particle size of the pore former. The addition of the polystyrene does significantly increase the pore volume and therefore the blood uptake with weight uptakes up to 50 μg in a similar sized probe to that previously in use combined with fast blood absorption but at the cost of a deterioration in mechanical properties. This is not unexpected as the mechanical strength of any ceramic material is inversely dependent on the porosity.

Thermogravimetric analysis shows that the polystyrene exhibits a single decomposition peak at 400° C. where it simply depolymerises. The phenolic decomposition of the novolak is more complex with two major decomposition processes at ˜360° C. and 520° C. and weight loss continuing up to 700° C. In a fully formulated pellet or other moulded structure containing both the resin and the polystyrene the main peak can be seen to be a superimposition of the two components. Differential scanning calorimetry shows a glass transition for the polystyrene at ˜70° C. but no melting point transition up to 300° C., whilst the phenolic resin has a glass transition at ˜130° C. and subsequently the main resin decomposition at 360° C. As in the context of commercial polymeric tips the macropore structure is the primary factor controlling both the amount of blood absorbed and rate of absorption. If the resin pellets are produced by tableting from the milled and classified precursor resin the pore volume should be 30-40%, fixed by the packing density of the powder. This volume and the pore size will be reduced if excess fines are present in the powder as particles of <˜20 μm can in principle fill the voids between the larger e.g. 70 μm particles. Conversely, if the primary particles do not pack properly in the cavity in the tableting press the pore volume will increase but in a random manner.

The macropore volume can as explained above be increased by the use of an additional pore former. Unlike the liquid pore former used to produce mesopores, this is achieved by the addition of polymer particles that, unlike the phenolic resin, decompose completely on pyrolysis to leave no residue and an additional void the size of each polymer particle. The particle size of the pore forming polymer is a critical parameter. If the pore former comprises small particles, the size of the cavities in the resin structure, no additional pores will be produced as the particles will simply infill the voids. This does however impact on the strength as the small particles inhibit sintering of the large resin particles. Conversely if the pore former particles are significantly larger than the primary particles these will expand the pore volume considerably but also significantly reduces the strength of the formed pellet. Even with a small concentration of the larger particles the connectivity between the resin particles is reduced. The preferred situation is for the pore former powder to be of a similar size to the precursor resin. Whilst the resin particle connectivity is reduced it is considerably better than with the large particles. In addition to the requirement for the pore former powder to leave no residue during pyrolysis it is also preferable that it does not melt prior to decomposition as this can reduce the pellet mechanical properties. It should also be easily milled to give the required particle size. As explained above, the preferred polymer is polystyrene although any other polymer meeting the above constraints could be used.

The method of production of the structured composite material can be by either extrusion or pressurised moulding. In the case of extrusion, the first component, preferably with a particle size between 20 and 100 μm is mixed with between 10 and 40% volume of the lignin binder powder (second component) along with the extrusion aids. The extrusion aids are well known to those skilled in the art but comprise primarily cellulose compounds such as Methocell, polyethylene oxide and other additives used to modify and control the rheology of the dough and water. The amount of water to be added depends on the porosity of the first component but should be sufficient to give a flexible dough.

The majority of the work on the production of pellets/tablets was carried out using a single station hand operated press. In this device the loose powder sample to be tableted was weighed into a compression chamber. Pressure was then exerted on the piston resulting in some compression of the sample. Whilst this methodology was very slow it allowed the formed pellets to characterised as a function of the pressure exerted on the tablet and the time it was held under pressure. The size (length) of the pellets could also be readily adjusted by the amount of sample added to the chamber. This device was used to establish the dependence of the pellet properties on the forming conditions. The work was subsequently transferred to a multi-station rotary tableting press to increase the throughput.

### Carbonisation and Activation

The transformation of the lignin bound structures into nanoporous structures is performed by carbonisation, i.e. high temperature treatment in an inert atmosphere and at temperatures from ˜500° C. upwards and where necessary activation. The pyrolysis process begins at about 400° C. and is largely complete by around 700° C. although further small weight losses continue up to around 1400° C. However, surface area development in the polymeric components is only significant above around 700° C. at which point the material is not strictly carbon but a pyropolymer. The inert atmosphere for pyrolysis can be secured by flowing suitable inert gas. Nitrogen and argon can be used as inert purge gases at any temperature whilst carbon dioxide is effectively inert up to around 800° C. in the absence of catalytic metals. Vacuum may also be used. Due to the presence of mesopores in these materials, which provide efficient escape routes for the volatile products, the heating rates employed can be very high—up to 10° C. per minute. The porosity of the carbon component can be further enhanced by conventional activation methods, e.g. by activation in carbon dioxide above 800° C., which can give surface areas as measured by BET 5 point method of up to 2000 m²/g or even up to 3000 m²/g. It has been found that “physical” activation with carbon dioxide at the temperatures in the range 850-900° C. gives rise predominantly to microporosity

Examination of the pyrolysed composite resin structures using scanning electron microscopy clearly shows the domains of the lignin derived binder and the second mesoporous resin phase. This confirms that the melt flow characteristics of the lignin are such that the lignin does not flow into the voids either between the particles or to the pores within the resin particles.

The walls of the resulting monolithic carbon have a structure with voids between the particles, the individual continuous void space leading into and through the monolith. The void structure in the walls of a monolith is controlled by the particles used to form the monolith. When the monolith is made from macro-particles with a mean particle size of D_{P} (FIG. 5) the macro pore size is typically 20% of the size of the precursor mean particle size. The sizes of the individual particles can be varied over a wide range from a maximum particle size of approximately 10% of the wall thickness, t, to a minimum practical particle size of about 10 μm. This gives a void size within the wall for a 1 mm wall thickness of 2-20 μm. The void size fixes the bulk diffusivity of the adsorbent molecules within the matrix. In embodiments the monoliths are square channel monoliths with a cell structure (cells per square cm) where the channel size is between 100 and 2000 μm and the wall thickness is between 100 and 2000 μm and with an open area of between 30 and 60% to give a good carbon packing density per unit volume and acceptable mass transfer characteristics.

How the invention may be put into effect will now be described by way of illustration only with reference to the following examples.

### Example 1 (Reference)

### Preparation of Phenolic Resin in Sinterable Powder Form

4 kg of a mixture of a standard medium molecular weight Novolac (Code J1098, supplied by Hexion Chemicals) in flake form was mixed with 200 g of HMTA. The mixture was jet milled to give a mean particle size of 40 μm. The powder was then placed in a tray and cured using a temperature ramp 3° C./min up to 100° C., dwell at 100° C. for 1 hour, then 3° C./min up to 150° C., dwell at 150° C. for 2 hours and then cooled back to room temperature. The cured block of resin was then hammer milled and jet milled to give a powder with a mean particle size of 40 μm.

### Example 2 (Reference)

### Preparation of Fully Cured Mesoporous Resin Powder

TE7/20 resin block was prepared as follows. 2841 g of ethylene glycol was mixed with 4315 g of 66.7% novolac resin in ethylene glycol after which HMTA (570 g) in 4276 g ethylene glycol was added to the mixture and stirred. The liquid mixture was then transferred to two metal trays. These were placed in the oven and ramped to 150° C. at 3° C./min at which temperature they were held for 2 hours. During curing ammonia was released which was trapped in a water scrubber. After curing the block of resin was granulated to give approximately 2 mm pieces which were suitable for water washing to remove the glycol. After washing and drying the resin granules were jet milled using a Hosokawa 100 AFG mill to give a powder with mean a particle size of 40 μm.

### Example 3

Carbon Structures from Partly Cured Novolak and Polystyrene

The porosity of the carbon structures e.g. tablets can be increased through the use of a polymeric pore former. This is a polymer that on pyrolysis has a zero-carbon yield. This pore former polymer may be milled to a particle size in the range of the size of the resin precursor powder. It is also preferred that this polymer does not melt before the onset of carbonisation of the resin powder. The preferred material is polystyrene and the material used in the present studies was supplied by Sigma Aldrich and had a MW of 35,000 although any material meeting the carbon yield requirement could be used. The polystyrene powder was thoroughly mixed with the resin powder and lubricant (1-2% stearic acid).

The results shown below are all based on a novolak precursor resin milled to 70 μm and classified to remove the fines smaller than ˜17 μm. The polystyrene powder used was milled to three sizes 17, 45 and 61 μm and was used unclassified at either 15% or 30% weight based on the resin powder.

As expected, the strength after pyrolysis at 700° C. was reduced with the extent of reduction dramatically impacted by the polystyrene level falling from 120 N in the absence of polystyrene to 15 N at 30% polystyrene. However, this was accompanied by an increase in the amount of blood wicked from ˜22 μg at 0% polystyrene to an average of ˜37 μg at 15% polystyrene and 42 μg at 30% polystyrene. There was a greater increase in wicking speed and an associated decrease in bubble formation with only a small amount observed after completion of the blood absorption. Based on the data below, the optimum polystyrene loading for a combination of blood uptake, speed of absorption, absence of bubble formation and strength was 15% wt with no major impact of polystyrene particle size. The higher blood capacity also then provides scope for further process enhancements to increase the mechanical properties. Higher polystyrene levels resulted in a reduction in the mechanical strength.

| | | PS | | | | wicking | | carbon |
|---|---|---|---|---|---|---|---|---|
| | | content/ | Carbon | bubble | wicking | weight | pellet | strength |
| Batch# | Sieving | size | Temp (C.) | formation | speed | μg | density | N |
| 10019 | sieved > | 0% | Si-500° C. | No | slow | 28.5 | 0.6 | 72 |
| | 53 micron | | | Bubbles | wicking | | | |
| | | | | | speed, | | | |
| | | | | | complete | | | |
| | | | | | wicking | | | |
| 10030 | unsieved | 0% | 950/2 | | | | 0.6 | 132 |
| 10031 | Classified > 17 | 30% | Cl-700° C. | Some Bubbles | Fast speed | 42.0 | 0.5 | 16 |
| | micron (mean 74) | (45 um) | | after complete | wicking, | | | |
| | | | | wicking | complete | | | |
| 10032 | Classified > 17 | 30% | Cl 500° C. | No | Fast speed | 51.8 | 0.5 | 11 |
| | micron (mean 74) | (45 um) | | Bubbles | wicking, | | | |
| | | | | | complete | | | |
| 10034 | Classified > 17 | 15% | Cl-700° C. | Bubbles after | Fast speed | 36.2 | 0.6 | 43 |
| | micron (mean 74) | (45 um) | | complete | wicking, | | | |
| | | | | wicking | complete | | | |
| 10039 | Classified > 17 | 15% | Cl-700° C. | Bubbles after | Medium speed | 39.3 | 0.62* | 45 |
| | micron (mean 74) | (61 um) | | complete | wicking, | | | |
| | | | | wicking | complete | | | |
| 10040 | Classified > 17 | 15% | Cl-700° C. | Bubbles after | Medium speed | 35.0 | 0.7 | 50 |
| | micron (mean 74) | (17 um) | | complete | wicking, | | | |
| | | | | wicking | complete | | | |

### Example 4

### Use of Hydroxy-Modifier to Enhance Sintering of Partially Cured Novolak Particles

For partly cured novolak resin particles, modification was made in the way in which the primary resin particles sintered during the tableting process. The glass transition temperature of the partially cured resin could be reduced through the addition of hydroxy modifiers. Water and ethanol are known to behave in this way, but their high volatility limits their usefulness. The ideal modifier remains in the formed resin tablets to the point where pyrolysis commences. The work to date has been based on ethylene glycol but other materials such as propylene glycol etc, could also be used.

To achieve the modification the formed resin tablets were soaked in an aqueous solution of the ethylene glycol. The pellets were then dried at ˜60° C. overnight which removed the water leaving a controlled level of glycol in the pellet. The glycol-containing pellet was then pyrolyzed in the normal way in either a nitrogen or carbon dioxide flow. In an initial test 60 tablets were soaked in a 50%water/50% EG solution for 3 hours. They were then dried at 60° C. overnight. The ethylene glycol treatment was carried out on the pellets produced from the sieved partially cured resin powder which the earlier studies had shown resulted in a reduced strength. The mercury porosimetry results shown below demonstrate that the sieving process has made a negligible difference to the carbon tablet density but that the EG treatment has resulted in a significant increase. By contrast the skeletal densities of the three materials, which reflects the underlying material properties only, are within experimental error identical. The strength of the EG modified sieved tablet had however increased by at a factor of ˜2.5

| | | Un- | Unsieved + |
|---|---|---|---|
| | Sieved | sieved | EG |
| Mercury data- | 0.7758 | 0.7633 | 0.8118 |
| tablet density | | | |
| g/ml | | | |
| Apparent | 1.1176 | 1.1105 | 1.1107 |
| skeletal | | | |
| density g/ml | | | |
| Crush strength | 120 | 105 | 250 |
| N | | | |

The mercury pore size distributions of the three materials were determined. The pore sizes of the two untreated materials were identical. In contrast the EG treated material showed a significant decrease in pore volume accompanied by a shift to a slightly smaller peak pore size as would be expected if the precursor particles had merged together slightly.

In a further study the above procedure was repeated but using tablets containing 30% polystyrene which reduced the untreated strength to 22 N. The EG solution concentration for the initial treatment was varied between 20 and 50%. The results shown below demonstrate a rapid rise in tablet strength with solution concentration reaching a peak at 100 N at 40% EG despite the use of the 30% polystyrene precursor.

10 discs of 10055 batch (30% PS) treated with various amount of EG in 50 ml solution

| | Average | Average | Average max | Average | Average | Average max |
|---|---|---|---|---|---|---|
| | length | diameter | applied compressive | length of | diameter of | applied |
| | of resin discs | of resin | load to | carbon | carbon | compressive load |
| | after drying | discs after | the resin | discs | discs | to the carbon |
| EG (%) | (mm) | drying (mm) | discs (N) | (mm) | (mm) | discs (N) |
| 0 | 3.65 | 6.06 | Greater than 300N | 3.6 | 5.07 | 25 |
| | | | (over loaded) | | | |
| 20 | 3.88 | 6.12 | Greater than 300N | 3.46 | 4.99 | 50 |
| | | | (over loaded) | | | |
| 30 | 3.95 | 6.11 | Greater than 300N | 3.46 | 4.96 | 90 |
| | | | (over loaded) | | | |
| 40 | 4.07 | 6.15 | Greater than 300N | 3.46 | 4.93 | 100 |
| | | | (over loaded) | | | |
| 50 | 4.17 | 6.20 | Greater than 300N | 3.29 | 4.90 | 97 |
| | | | (over loaded) | | | |

The effect on the blood properties is shown in the table below:

| | | | wicking | wicking | drain- |
|---|---|---|---|---|---|
| % PS | rod post | | time | volume | age |
| level | treatment | strength | time s | μg | 0-5 |
| 0 | NO | 41 | 5 | 38.1 | 3 |
| 15 | NO | 29 | 14 | 45.1 | 2 |
| 15 | 50% EG | 81 | 5 | 38.3 | 2 |
| 30 | 80% EG | 54 | 3 | 44.6 | 1 |

It can be seen that after the EG treatment the pellets with 15% polystyrene now μg show good strength and a good wicking time combined with a good blood adsorption rate and capacity combined with good leakage properties. With the 30% polystyrene tablets, which would normally exhibit severely degraded mechanical properties, the tablets after treatment with an 80 wt EG/water solution now show excellent blood volume and rate properties combined with good strength and leakage properties. These results demonstrate that preferred production conditions to achieve better than the target performance requirements comprise a polystyrene level of 15 to 30% combined with an ethylene glycol post-treatment with between 50 and 80% EG/water solutions.

Pellets or tablets produced as described above may be treated to make them more hydrophilic by plasma treatment or a plasma enhanced chemical vapor deposition (PCVD) e.g. using a machine from PVA TePLA (see e.g. U.S. Pat. No. 6,943,316 Konkavo et al., TePLA AG).

### Example 5

### Trimodal Pore Systems

The materials described in WO 2017/009662 (Tennison) and US 2019/0022623 (US 15/745399) have a more complex micro/meso/macro trimodal structure which requires a more complex production route. The reason for the introduction of the mesopores is that these can adsorb large molecules such as proteins and cytokines allowing some fractionation of the blood absorbed in the pellets. Once the blood has filled the macropores by capillary suction the smaller molecules such as the proteins can adsorb into these meso pores.

The production route is summarised below and differs from the routes described above in that ethylene glycol cannot be used to enhance the sintering properties of the precursor resin particles. The highly mesoporous resins have to be carefully treated at step (b) below to remove the ethylene glycol used in this route as a meso/macro pore former as this plasticizes the porous resin and can lead to a collapse of the mesopore structure during the subsequent pyrolysis stage.
(a) The precursor novolak resin and the hexamine curing agent were dissolved in ethylene glycol (EG) where the EG functions as a pore former. The size and volume of the meso/macropores were fixed by the ratio of EG to the total solids (novolak+hex). This solution was poured into trays and cured at 150° C. for 1 hour. A critical difference between the two routes was the amount of hex used in this route. The EG solvent inhibits curing and it was necessary to use typically 20 parts hex to achieve a stable meso/macropore structure. This represented complete resin cure as against the partial cure used in the conventioal route using non mesoporous resin.
(b) The block of cured resin containing the EG is then crushed to give ˜2 mm pieces and the EG pore former is removed either by hot water washing or vacuum drying. It is essential that the EG is removed at this stage as the presence of the ethylene glycol lowers the glass transition temperature of the resin matrix and the pore structure within the cured resin particles will then collapse during carbonisation.
(c) The dried resin can then be milled to the particle size required for the finished pellets e.g. 70 μm and classified to remove fines larger than ˜17 μm or <20 micron fines.
(d) Polystyrene powder may be added milled e.g. to 60 or 70 μm and may be used unclassified at e.g. 15% or 30% weight based on the resin powder.
(d) This fully cured resin, which cannot sinter, then requires the addition of a binder to produce stable monolithic structures. Lignin, preferably Organosolv lignin is used, but the amount should be minimised (10-20 parts weight based on the weight of the novolak) as higher levels will result in an unacceptable reduction in macroporosity. The primary carbon particles have a bimodal pore structure comprising the same micropores as the materials from Example 1 but also now contain meso/small macro pores (2 nm to 100 nm) introduced by the ethylene glycol pore former. The powder to be pelleted may comprises 80 parts cured resin powder, 20 parts lignin, 20 parts polystyrene and 2% wt stearic acid.
a) The pellets are then carbonised at 500° C. to 800° C. in nitrogen or carbon dioxide, preferably carbon dioxide.

The monoliths produced by this route comprise the primary particles with <2 nm micropores and meso/macropores with a pore size of 2 to 100 nm where the mesopore size was fixed by the EG:solids ratio in (a) above and macropores between the primary particles resulting from the polystyrene particles.

It is also expected that this production route using the lignin binder could be applied to the production of the bi-modal pore structure tablets in place of the ethylene glycol sintering modification route described above.

Tablets produced as described above may also be treated to make them more hydrophilic by plasma treatment or a plasma enhanced chemical vapor deposition (PCVD) e.g. using a machine from PVA TePLA (see e.g. U.S. Pat. No. 6,943,316 Konkavo et al., TePLA AG).

1. A process for producing a structure into which blood or other bio-fluids can flow by capillary action, said process comprising:
mixing particles of novolak resin and particles of hydrocarbon polymer;
producing an uncarbonized structure from the mixture by pressurised moulding; and
carbonizing the moulded structure, the hydrocarbon polymer being a polymer that on pyrolysis has a zero carbon yield, and the particles of the hydrocarbon polymer leaving voids in the carbonized structure of sufficient size for flow of whole blood into and through the structure to be enhanced.

2. The process of claim 1, wherein the particles of novolak resin are of mean size 40-70 μm and have been classified to remove powder of size less than 20 μm.

3. The process of claim 1, wherein the particles of hydrocarbon polymer have a mean particle size of 40-70 μm.

4. The process of claim 1, wherein the hydrocarbon polymer is polystyrene. 20

5. The process of claim 4, wherein the polystyrene has a molecular weight of 30,000-40,000.

6. The process of claim 1, wherein the particles of novolak, hydrocarbon polymer and binder are mixed with a lubricant.

7. The process of claim 6, wherein the lubricant is stearic acid.

8. The process of claim 1, wherein the mixture comprises 80 wt parts cured novolak, 20 wt parts lignin, 20 wt parts polystyrene and 2 wt parts stearic acid

9. The process of claim 1, wherein the particles are of partly cured and milled novolak resin, the novolak particles when in the moulded structure not exhibiting bulk flow during carbonization but sintering at inter-particle contact points during carbonization to provide a consolidated structure.

10. The process of claim 9, wherein the partly cured particles are treated prior to moulding with ethylene glycol or another polyol sintering aid

11. The process of claim 1, wherein the particles are of fully cured and milled novolak resin, and are mixed with the hydrocarbon polymer, the lubricant and with a binder for providing a consolidated structure.

12. The process of claim 11, wherein the milled particles are the result of:
(a) dissolving a water insoluble lignin and novolak resin in ethylene glycol;
(b) dissolving hexamethylenetetramine (HMTA) curing agent in ethylene glycol;
(c) mixing the two solutions to give at least 15 parts of HMTA per 100 parts of lignin+novolak, the ratio of the lignin+novolak+HMTA to ethylene glycol and of lignin to novolak being selected according to required macroporosity.

13. The process of claim 12, wherein the mixed solution is poured into a tray and cured at 150° C. for 2 hours.

14. The process of claim 13, wherein the cured resin is granulated to form particles of size approximately 2 mm, after which the ethylene glycol is removed from the granulated cured resin by either washing in hot water or vacuum drying and the ethylene glycol-free granulated resin is jet milled to provide a powder of which at least 90% is of size <100 μm.

15. The process of claim 1, wherein after moulding the structure is carbonized at a temperature of at least 500° C., in an inert gas.

16. The process of claim 1, wherein after moulding the structure is carbonized at a temperature of 500° C. to 800° C. in nitrogen or carbon dioxide and optionally activated by treatment in flowing carbon dioxide at a temperature of 800° C. for a time sufficient to give a weight loss of up to 20%.

17. The process of claim 1, wherein carbonization of the moulded structure results in an open-celled porous carbonized material shaped as an absorbent probe.

18. The process of claim 17, wherein the probe it is of sufficient size and porosity to absorb for analysis about 1 μl to about 100 μl of whole blood in about 2-5 seconds without separating the blood from plasma.

19. The process of claim 17, wherein the resulting probe has a length of less than 5 mm, a cross-sectional area of less than 20 mm² and a flat surface that in use is exposed and available for placement against a fluid sample on a surface to absorb the sample .

20. The process of claim 17, wherein the probe has any of the following features:
(a) it is suitable for use with devices that can manipulate a pipette tip;
(b) it has a pore volume of at least 35%;
(c) it is of size sufficient to absorb about 5-20 μl of blood.
